(19) United States
(12) Patent Application Publication
(10) Pub. No.: US 2004/0175490 A1
(43) Pub. Date: Sep. 9, 2004

(54) Hybrid method for the production of carriers for analyte determination

(76) Inventors: Cord F Stahler, Weinheim (DE); Peer F. Stahler, Mannheim (DE); Markus Beier, Heidelberg (DE); Anke Wixmerten, Rheinstrasse (DE); Ralf Mauritz, Frankfurt am Main (DE); Andrea Schlauersbach, Darmstadt (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/477,282

(22) PCT Filed: May 10, 2002
(86) PCT No.: PCT/EP02/05179

(30) Foreign Application Priority Data
May 9, 2001 (DE) 101223579

(51) Int. Cl. B05D 3/00; C07H 21/04; C07K 14/00

(52) U.S. Cl. 427/2.11; 530/350; 536/23.1

(57) ABSTRACT

The invention relates to a method and a device for the production of a carrier, in particular a microfluidic carrier, for determining analytes.

The invention relates to a method and a device for the production of a carrier, in particular a microfluidic carrier, for determining analytes.

In recent years, a valuable means was generated in the form of the technology of receptor arrays immobilized on a carrier, for example DNA chips, which allows carrying out complex analyte determinations in a rapid and highly parallel manner. The biophysical principle on which the receptor arrays are based is that of the interaction of a specific immobilized receptor with an analyte present in a liquid phase, for example by nucleic acid hybridization, with a multiplicity of receptors, for example hybridization probes, being arranged on various regions of the carrier, which receptors specifically bind in each case to various analytes present in the sample, for example complementary nucleic acid analytes.

In order to be able to process complex biological problems such as gene expression studies, target validation, sequencing or resequencing reactions by means of receptor arrays, for example DNA chips, an efficient production of high-quality receptor arrays is of fundamental importance. To this end, DNA arrays may be produced by the spotting technology (Cheung et al., Nature Genet. Suppl. 1999, Vol. 21, 15-19) and additionally also in situ by using phosphoramidite synthetic building blocks (Caruthers et al., Tetrahedron Lett., 1981, 1859). In this case, distinction can be made between wet-chemical methods (Maskos et al., Nucleic Acids Res. 1992, Vol. 20, 1679-1984) and photochemical methods (Pease, Proc. Natl. Acad. Sci., 1994, Vol. 91, 5022-5026).

For example, WO 00/13018 describes a carrier and a method for analyte determination which allow integrated receptor synthesis and analysis. There, the receptors are preferably synthesized using photoactivatable receptor building blocks. Alternatively, a synthesis of receptor building blocks by wet-chemical methods is also disclosed.

U.S. Pat. No. 6,022,963 discloses a variant of the photochemical methods. This publication is concerned with novel photochemical protective groups. Novel photochemical compounds and the principal possibility of removing said photochemical protective groups also by wet-chemical steps are described. In a variant thereof, the method is modified in such a way that initially wet-chemical protective groups are provided on the receptor building blocks, which are then removed in order to replace them in situ on the entire carrier with photochemical protective groups. This is then followed again by a photochemical step which makes possible the desired space-resolved synthesis. A combination of photochemical and wet-chemical receptor building blocks is not disclosed.

Wet-chemical methods use receptor building blocks, for example phosphoramidite nucleotide building blocks, which carry a temporary wet-chemical protective group in the 3′ or/and 5′ position. This protective group is removed by a wet-chemical step, for example by acid treatment, for example with trichloroacetic acid, in the case of the common dimethoxytrityl protective group. A problem is the two-dimensional application of the deprotection medium (acid) so that a space-resolved removal is possible only with great difficulty and individual regions on the carrier can hardly be addressed. Advantageously, however, abstraction of the temporary protective group by wet-chemical methods and condensation of the next synthesis building block are highly efficient (a.≧98-99%).

Photochemical methods use receptor building blocks, for example phosphoramidite nucleotide building blocks, which carry a temporary photochemical protective group in the 3′ or/and 5′ position. This protective group is removed in a photochemical step. This may be carried out by location-specific illumination of the region from which the photoprotective group is to be removed and in which the receptor synthesis is then to be continued. Here, the deprotection medium (light) may be applied not two-dimensionally but in a space-resolved manner, for example by using a mask or via a programmable light source (see, for example, WO 00/13018). Thus, individual locations of synthesis can be addressed. Disadvantageously, however, the temporary photoprotective group is removed with comparatively low efficiency (approx. 90-95%), and thus the quality of synthesis and overall yield are reduced compared to wet-chemical methods.

It was an object of the present invention to provide a system for the production of a carrier for analyte determination, which system avoids, at least partially, the disadvantages of the prior art.

This object is achieved by a method which combines the advantages of wet-chemical and photochemical processes. These two methods acting specifically in combination result in novel strategies for the construction and application of receptor arrays, for example DNA chips, such as, for example, arrays with a plurality of different receptor sequences per position or region which were previously not possible using either of the two methods alone.

The hybrid method of the invention uses both wet-chemical and photochemical receptor building blocks, for example phosphoramidite building blocks.

The synthetic route may be optimized using a specific algorithm which ensures that the receptors to be synthesized on the array can be synthesized as quickly as possible and with high quality. The algorithm can be extended beyond the use of two different receptor building blocks, for example photolabile and acid-labile receptor building blocks, by using further protective groups, for example space-resolving protective groups such as, for example, electrochemical protective groups or/and other wet-chemical protective groups (e.g. base-labile or oxidation-labile protective groups) so as to still further increase the flexibility of the system.

The present invention thus relates to a method for producing a carrier for the determination of analytes, comprising the steps:

(a) providing a carrier,

(b) conducting a liquid containing building blocks for the synthesis of polymeric receptors over the carrier,

(c) immobilizing in a location- or/and time-specific manner the receptor building blocks in in each case predetermined regions on said carrier and

(d) repeating steps (b) and (c), until the desired receptors have been synthesized in the in each case predetermined regions,

characterized in that

synthesis of said receptors comprises a combination of wet-chemical and photochemical synthesis steps.

The present invention is particularly distinguished by the possibility of integrating the method for the production of the carrier with a detection system for analyte determination. Said detection system may be used for integrated synthesis and analysis, in particular for constructing complex carriers, e.g. biochips, and for analyzing complex samples, e.g. for genome analysis, gene expression analysis or proteome analysis.

The recep tors are synthesized in situ on the carrier, for example by conducting fluid containing receptor synthetic building blocks over the carrier, immobilizing said building blocks in the in each case predetermined regions on the carrier in a location- or/and time-specific manner and repeating these steps until the desired receptors have been synthesized in the in each case predetermined regions on the carrier. An essential feature of the receptor synthesis of the invention is the combination of at least one wet-chemical synthesis step and at least one photochemical synthesis step. The receptor synthesis furthermore comprises preferably an online process monitoring in order to guarantee an adequate quality of the receptors immobilized on the array.

The carrier produced by the method of the invention is preferably integrated in a device for determining analytes, which comprises

(i) a light source matrix, preferably a programmable light source matrix, e.g. selected from a light valve matrix, a mirror array and a UV laser array,

(ii) a carrier, preferably a microfluidic carrier with channels, in particular with closed channels, which contain the predetermined regions with the in each case differently immobilized receptors, said channels being preferably in the range from 10 μm to 10 000 μm, particularly preferably in the range from 50 to 250 μm, and in principle being designed in any possible form, for example with round, oval, squared or rectangular cross section,

(iii) means for supplying fluid to the carrier and for discharging fluid from the carrier and

(iv) a detection matrix, for example an optical detection matrix such as, for example, a CCD matrix or/and an electronic detection matrix as described in WO 00/13018.

In a preferred embodiment, the carrier provides, by way of a division into fluidic subspaces which can be addressed independently of one another, the possibility of determining location-specific immobilization. WO 00/13018 describes a carrier fulfilling this criterion. In this context, the carrier provides division of the reactive regions into 2 or more subspaces.

The receptors are preferably selected from biopolymers which can be synthesized in situ on the carrier from the appropriate synthetic building blocks by means of a combination of light-controlled and wet-chemical processes. Synthetic building blocks which may be used are both monomeric, for example mononucleotides, amino acids, etc., and oligomeric building blocks, for example di-, tri- or tetranucleotides, di-, tri- or tetrapeptides, etc. The receptors are preferably selected from nucleic acids such as DNA, RNA, nucleic acid analogs such as peptide nucleic acids (PNAs), proteins, peptides and carbohydrates. The receptors are particularly preferably selected from nucleic acids and nucleic acid analogs and used in a detection method for hybridization of complementary nucleic acid analytes.

The receptor synthesis preferably comprises using synthetic building blocks with wet-chemical protective groups and receptor building blocks with photochemical protective groups. It is also possible, where appropriate, to use synthetic building blocks which carry both wet-chemical and photochemical protective groups or hybrid protective groups, i.e. groups which can be removed in two stages via a wet-chemical and a photochemical step. Examples of wet-chemical protective groups are any protective groups, as known from the prior art, for synthesis of biopolymers such as, for example, nucleic acids or peptides, on solid carriers. Preferred examples are acid-labile protective groups, base-labile protective groups, protective groups labile to oxidation or enzymically removable protective groups. Particular preference is given to using acid-labile protective groups such as dimethoxytrityl, for example. Any photochemical protective groups, as known from the prior art for synthesis of biopolymers such as, for example, nucleic acids or peptides, on solid carriers, may be used for the photochemical synthetic steps. Preferred examples of photochemical protective groups are described in DE 101 05 079.8, and preferred examples of hybrid protective groups are described in DE 101 05 077.1.

Further preferred protective groups are “two-stage” protective groups which are activated by an illumination step and then cleaved by a chemical treatment step. The chemical treatment step preferably comprises a treatment with base, a treatment with acid, an oxidation, a reduction or/and an enzymic reaction. Particular preference is given to derivatized trityl groups as two-stage protective groups, as described in DE 101 32 025.6.

The present invention furthermore comprises using wet-chemical protective groups, for example acid-labile protective groups such as trityl protective groups, the reagent required for removing the protective group, which reagent may be, for example, an acid such as trichloroacetic acid, being formed in situ by illuminating an acid precursor, for example a trichloroacetic ester of substituted o-nitrobenzyl alcohols. Examples of procedures of this kind have been described by Serafinowski and Garland at the “Chips to Hits 2001” conference (IBC's 8th Annual International Microtechnology Event) from 10.28-11.01.2001.

The method of the invention preferably comprises the production of a carrier with a plurality of, preferably with at least 50 and particularly preferably with at least 100, different receptor regions which are capable of reacting with in each case different analytes in a single probe. The method of the invention may be used for producing carriers, the receptors in each region of said carrier containing only a single sequence of building blocks. In another embodiment, however, the method of the invention may also be used for producing carriers, with the receptors containing in at least one region of said carrier a plurality of different sequences of building blocks.

The synthesis may commence by either a wet-chemical or a photochemical step.

Preferred embodiments of the method of the invention will now be illustrated below:

Hybrid Method For Synthesizing One Sequence Per Position

In order to be able to generate a multiplicity of different receptor sequences on the carrier as efficiently as possible, the synthesis strategy (sequence of condensation steps) is iterated with respect to as high a proportion as possible of wet-chemical synthone building blocks using a computer program. The iteration is taken over by a specifically developed algorithm. As the boundary condition, the aspect that all sequences need to be provided in a space-resolved manner is taken into account. However, it is not necessary here that, in the case of condensation of wet-chemical synthone building blocks, the latter must be located on the identical synthetic level. The shortest synthetic route is preferably calculated according to the following plan:

1. Definition

Synthetic route refers to the sequence (with repeats) of nucleotides which is required in order to generate completely all receptors of the set of receptors.

2. Boundary Conditions

In order to be able to generate completely a set of receptors of size s with a maximum length of n receptor building blocks, at least n synthetic cycles are required. This is the case, for example, when all receptors have the same sequence, for example base sequence. If the receptors are generated in layers, i.e. all receptors must have reached the same length m before being extended to the length m+1, a maximum of 4 cycles per layer and thus 4ⁿ cycles in total are required.

The number of synthetic steps is thus always limited by the length of the receptors and not by the number of receptors to be generated. There are 4ⁿ possible sequences in which the nucleotides can be coupled in order to generate a set of receptors of the length n.

3. Calculation of the Shortest Route

In order to determine the shortest synthetic route, firstly suitable boundary conditions are determined, such as, for example maximum and minimum length of the route to be calculated. This is followed by running an iteration of all possible combinations and selecting the shortest one or, in the case of several shortest routes, one which is to be used for synthesis. Choosing additional boundary conditions makes it possible to stop the iteration in time, when it is no longer possible for the sequence observed to be shorter than the one calculated previously.

4. Determination of the Types of Deprotection in the Individual Synthetic Steps

With the shortest synthetic route being known it is then possible to determine which protective groups are to be carried by the individual building blocks. Each building block which is followed by that and only that building block to be coupled next in the synthetic sequence can be coupled with a wet-chemical, for example acid-labile, protective group. If a coupled building block is followed by different building blocks, the former must carry a photolabile protective group in order to be able to continue synthesis in a location-specific manner.

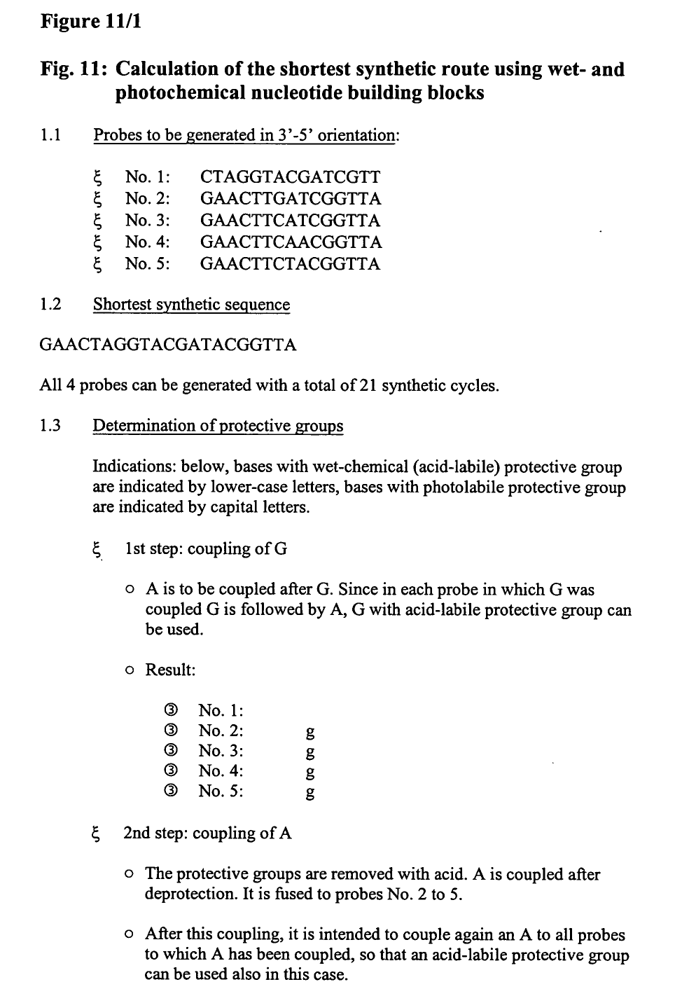

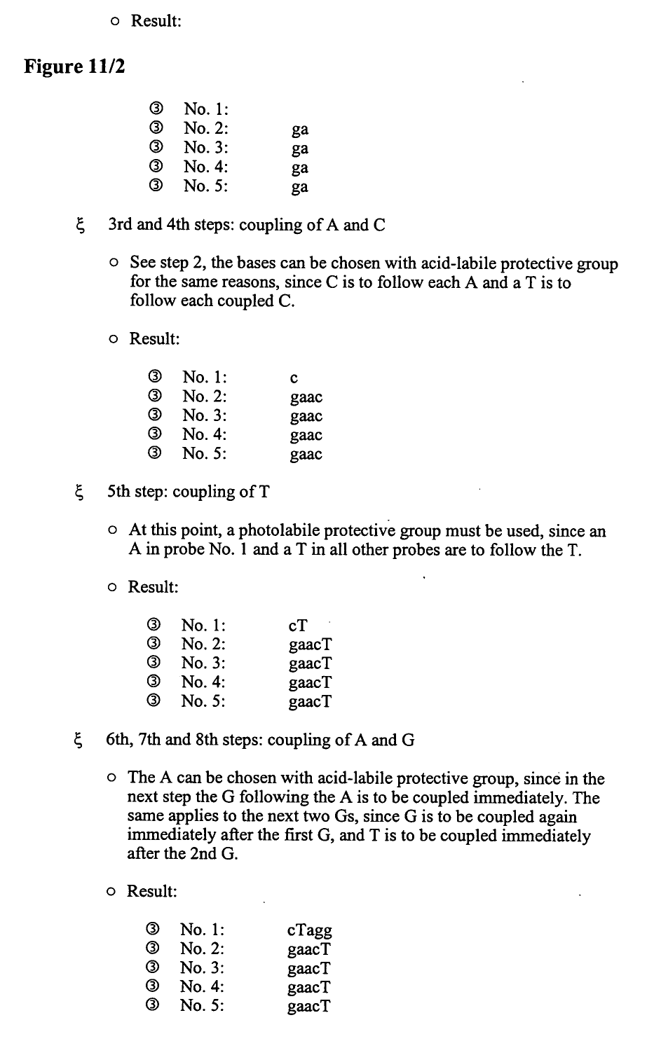

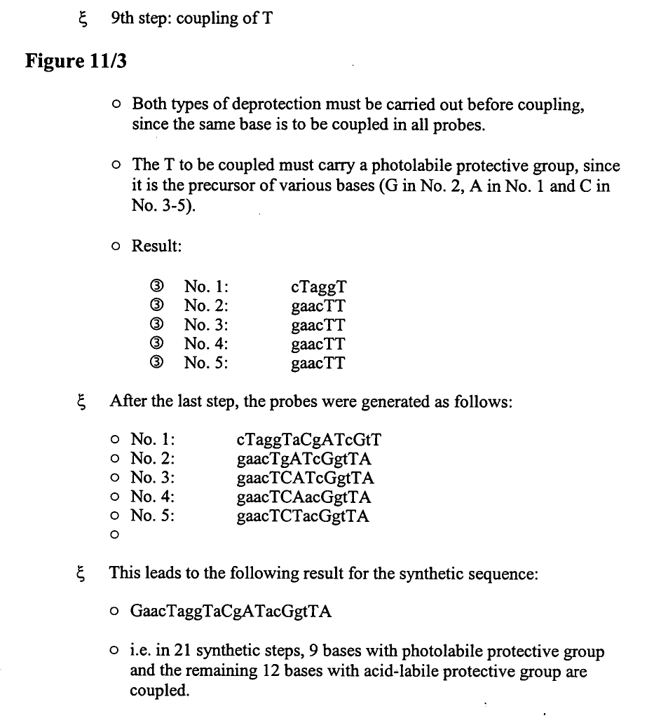

FIG. 11 depicts a specific exemplary embodiment.

In a particularly preferred embodiment, the hybrid method of the invention uses a microfluidic reaction carrier, as is described in international patent application WO 00/013018. This results in an additional spatial separation of the synthesis sites, which may be used for increasing the efficiency of synthesis. In this connection, the computer-assisted optimization of the synthesis strategy, where as many wet-chemical synthetic building blocks as possible are to be used, additionally also includes in this evaluation the microfluidic channels in which condensations of identical synthetic building blocks are combined.

According to the invention, it is also possible to provide a plurality of sequences per position by the above-described hybrid method. To this end, one embodiment—at least one step—comprises according to the invention reacting wet-chemical and photochemical synthone building blocks simultaneously.

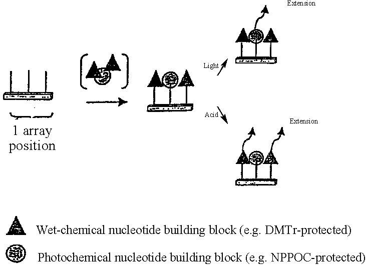

This leads to either one portion of the molecules per position, after applying a wet-chemical deprotection medium (e.g. acid), or another portion, after application of a photochemical deprotection medium (light), then being synthetically extendable. The synthone building blocks depicted below in a general embodiment may serve as an example:

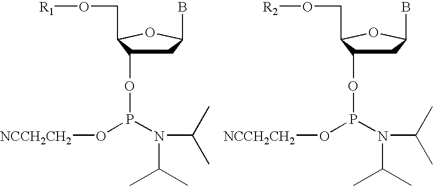

R₁ and R₂ are protective groups in each case orthogonal to one another, i.e. protective groups which can be removed under in each case different conditions. Examples of such a combination of R₁ and R₂ are DMTr (acid-labile) and NPPOC (photolabile) protective groups. It is possible, after wet-chemical or photochemical deprotection, to adjust the particular proportion of the probes extendable in the next step per position via the mixing ratio used for reacting the synthones.

In another embodiment, provision of a plurality of sequences per position is achieved using special hybrid building blocks (A), (B) and in particular (C), as have been described previously in German patent applications DE 100 41 539.3 and DE 100 41 542.3.

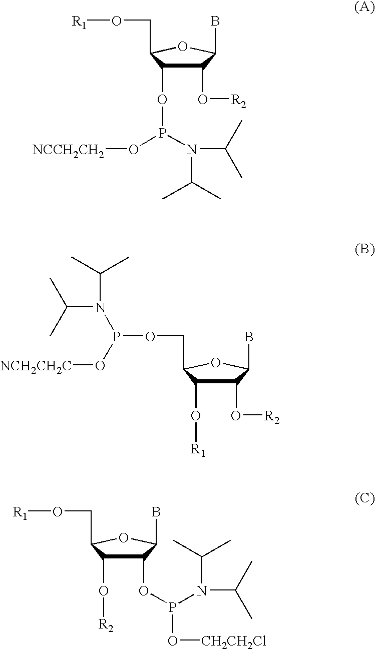

R₁ and R₂ are protective groups in each case orthogonal to one another. Here, condensation of the hybrid branching building block takes place at least once per array synthesis. If said branching building block is applied several times during the array synthesis, it is thus possible to construct dendrimeric structures. The principle of the hybrid branching building block is depicted below:

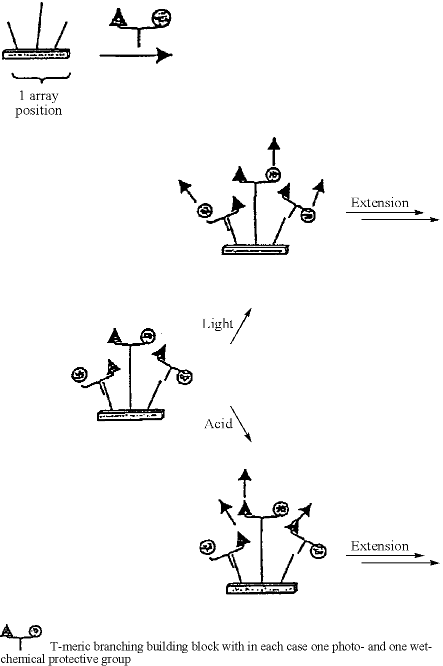

In the case of the hybrid building block, the presence of wet-chemical and photochemical protective groups in a single molecule is used, for example, for continuing synthesis after wet-chemical deprotection on one end of the molecule and then inducing, at a later time, continuation at the other end of the hybrid building block. Using this method, it is possible to construct a plurality of sequences per position. Depending on the use of hybrid branching building blocks of type (A), (B) or (C), the chain extensions occur at different sites:

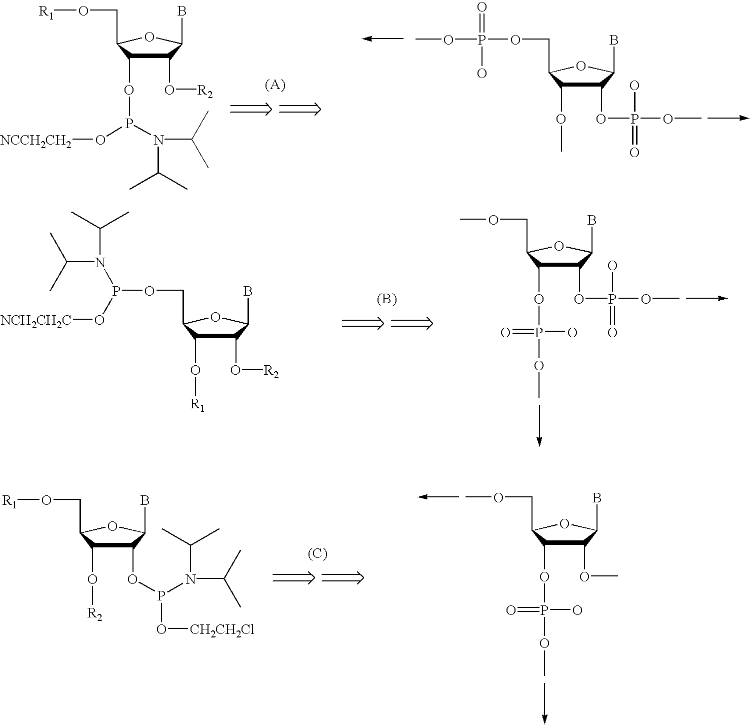

Any nucleotide building blocks (e.g. 2′-, 3′-, 5′-phosphite amides) may be used for chain extension, possibly resulting in 3′-5′, 5′-3′, 5′-5′, 3′-3′, 2′-2′, 5′-2′, 3′-2′, 2′-3′ or 2′-5′ chain extensions at the branching building block.

A specific procedure is depicted below by the example of a hybrid branching building block of type (A). After removing protective group R₁ (e.g. photolabile), the chain is extended at the 5′ end of the branching building block and sequence 1 is generated. After completion of sequence 1, further growth is prevented by a capping step. If the R₂ protective group (e.g. acid-labile) is then removed, the chain can be extended at the 3′ end, until construction of sequence 2 is complete. Sequences 1 and 2 may but need not be complementary to the identical target sequence present in the sample to be investigated.

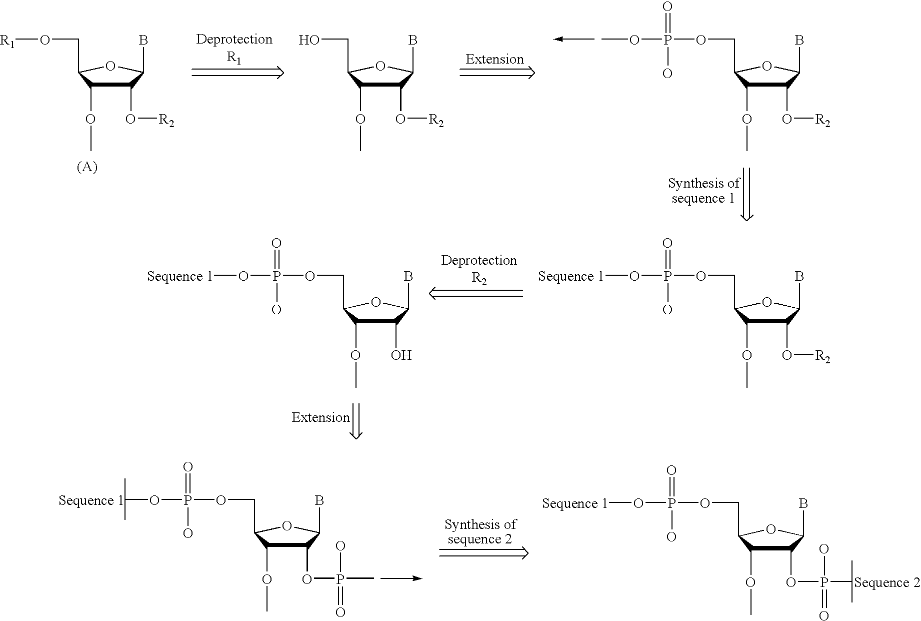

If, for example, the second sequence constructed in addition to the probes required for the actual hybridization experiment is the same sequence in all positions, this sequence may serve as reference sequence. This makes it possible to normalize the microarray very accurately, since there is a control sequence for each position.

In order to prevent the reference probes from producing increased background signals, it must be ensured that the sample to be investigated does not contain these sequences. According to the invention, this is achieved by doing this in the run-up to the array production or/and by using particular building blocks of nucleic acid analogs which do not pair with DNA molecules (Beier et al., Science 1999, Vol. 283, 69-703) for generating the reference probes.

For analysis, the DNA targets to be investigated and the corresponding complementary reference sequences—which pair only with the same kind of sequences and not with the DNA to be investigated—are then hybridized on the microarray, either simultaneously (e.g. by 2 color detection) or separately. Thus it is possible for each position of the array to individually normalize the hybridization signal of the sample sequence to be investigated to the signal of the reference sequence located on the same position. This makes it possible to average out irregularities of the array due to production (irregular illumination, irregular derivatization) or hybridization (fluff, reflections).

In another embodiment, it is possible to use hybrid branching building blocks in enzyme reactions. Thus, for example, a hybrid branching building block may be used as primer for a polymerase reaction or else a ligase reaction.

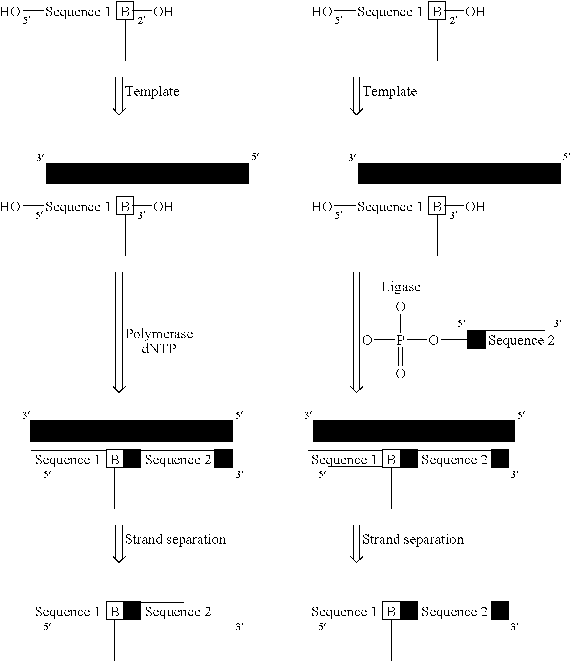

In this connection, the attachment site required for the enzyme need not necessarily be located directly at the hybrid linking building block (as depicted above).

There may quite possibly be a plurality of nucleotide building blocks between linking building blocks and the enzyme attachment site.

The present invention allows considerable improvements in the synthesis of receptor arrays compared to the known methods, since the hybrid method can employ a computer-optimized synthesis strategy for space-resolved in situ synthesis. As many wet-chemical synthone building blocks as possible are used here, in order to achieve synthesis products of higher quality. In addition, the use of as many wet-chemical building blocks as possible also allows an increase in the rate of synthesis, without restricting the flexibility of the space-resolved synthesis. Optimization of the synthetic route can be calculated using a specific algorithm, as indicated above.

The combination of wet-chemical and photochemical building blocks makes it possible to generate a large number of different receptors in a highly parallel way in the in situ synthesis of receptor arrays. This may be increased still further by using a microfluidic reaction carrier, for example with a multiplicity of parallel channels.

Furthermore, parallel condensation of wet-chemical and photochemical receptor building blocks enables the specific construction of a plurality of receptor sequences per position or region. This may also be carried out by using hybrid building blocks which carry both a wet-chemical and a photochemical protective group. Thus it is possible, for example, for one sequence per position to serve as reference (quality control), while another one is available for the actual experiment.

The improvements mentioned cannot be achieved solely by a pure wet- or photochemical method for the production of the carrier.

The following figures are intended to further illustrate the present invention.

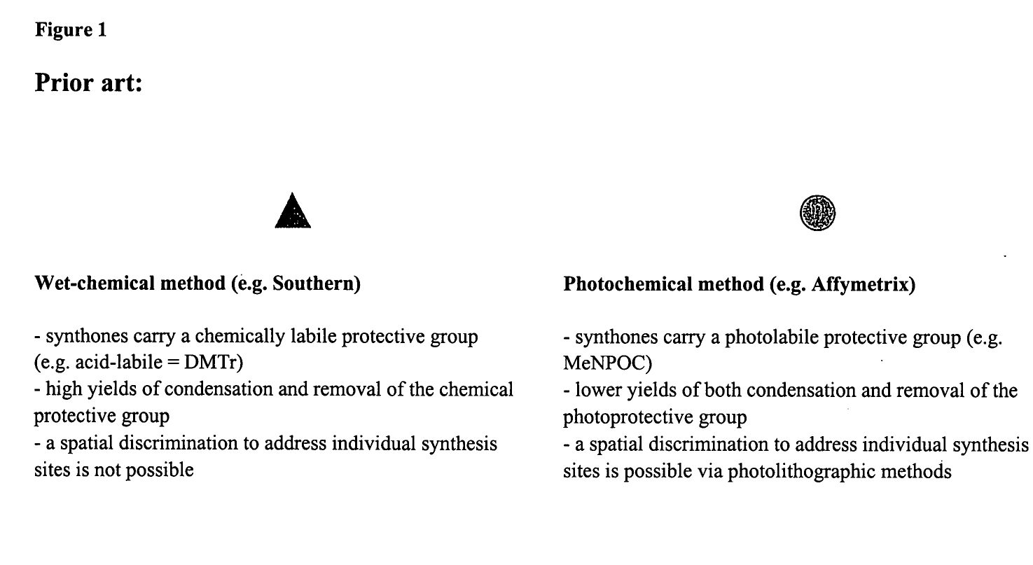

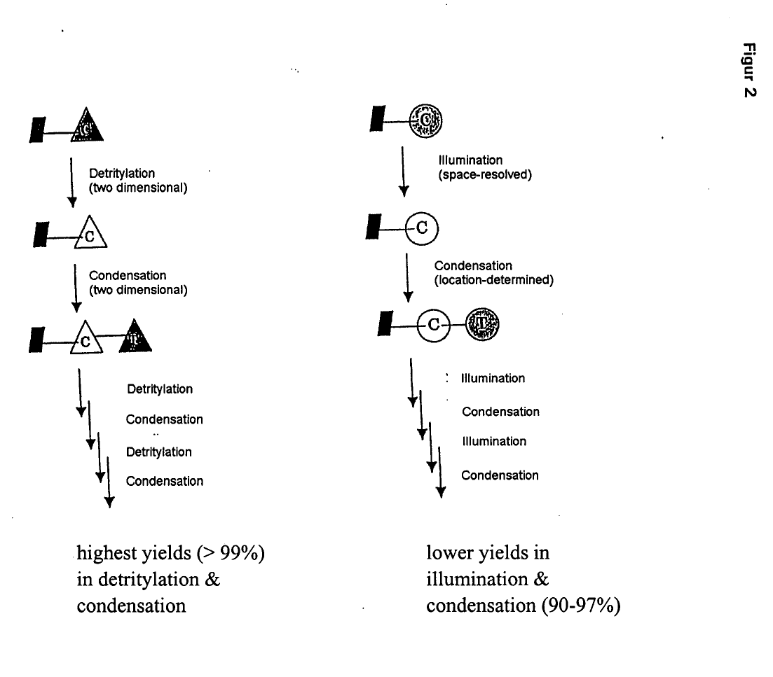

FIG. 1 shows a comparison of wet-chemical and, respectively, photochemical methods for generating DNA arrays by means of in situ synthesis (prior art)

FIG. 2 shows a comparison of the reaction processes in wet-chemical and photochemical methods (prior art)

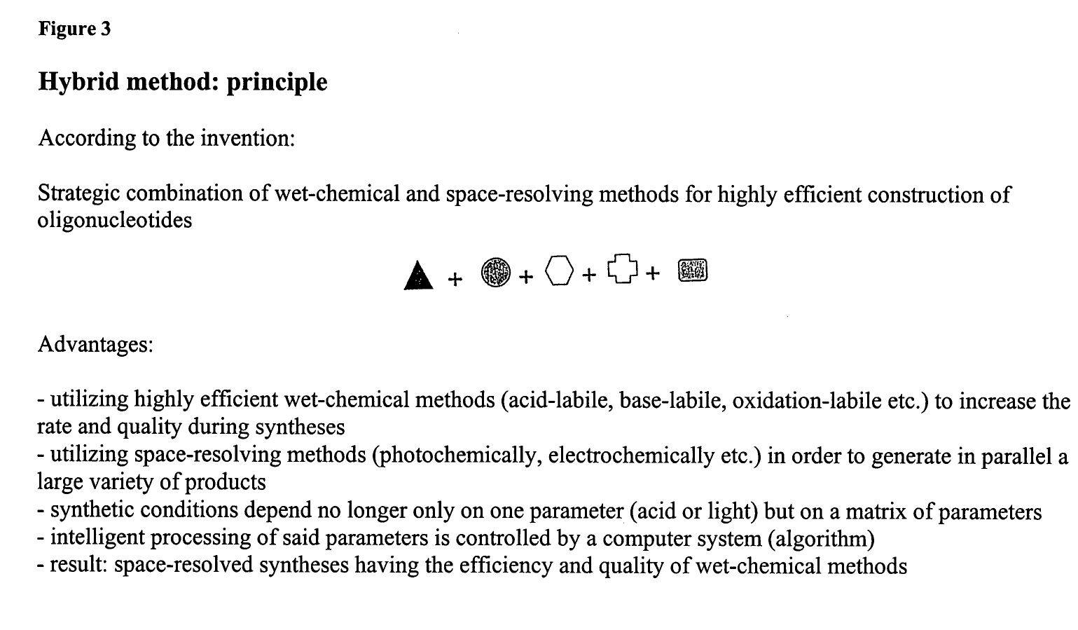

FIG. 3 shows the principle of the hybrid method with synthesis of one sequence per position

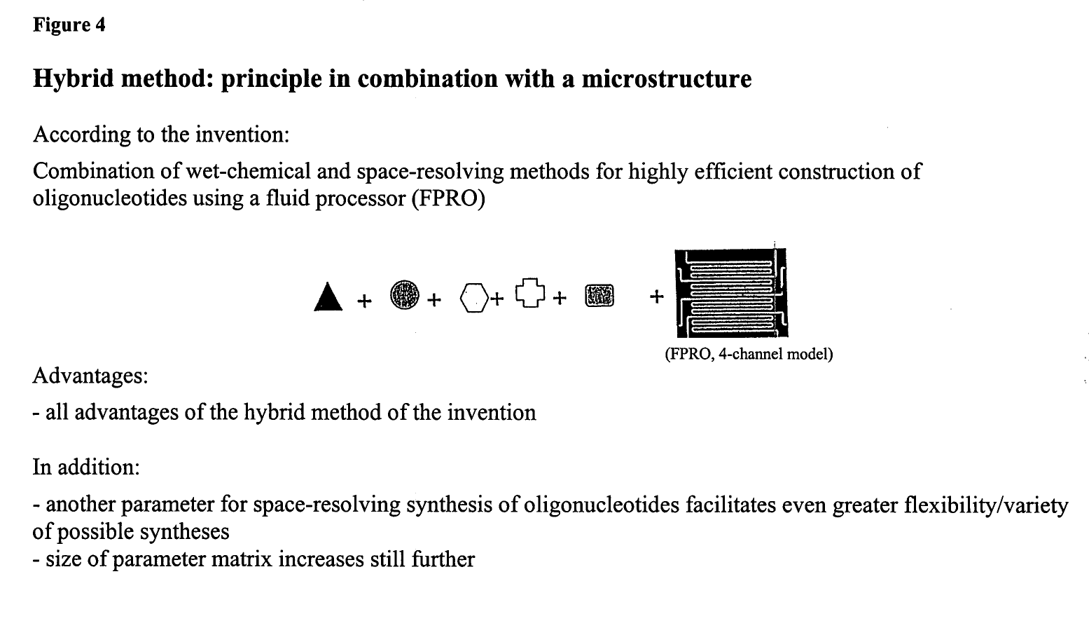

FIG. 4 shows the principle of the hybrid method with synthesis of one sequence per position, using a microfluidic reaction carrier

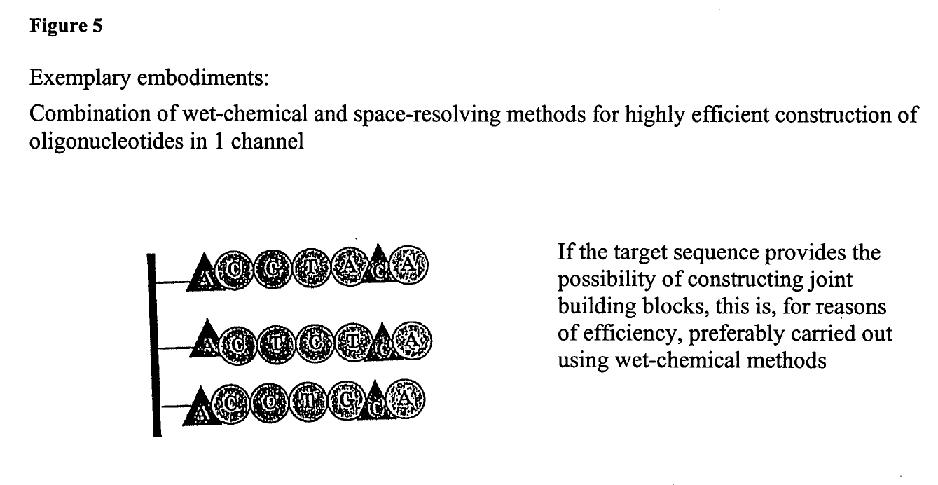

FIG. 5 shows an embodiment of the inventive combination of wet-chemical and photochemical methods; the jointly fused wet-chemical building blocks are located on one synthetic level

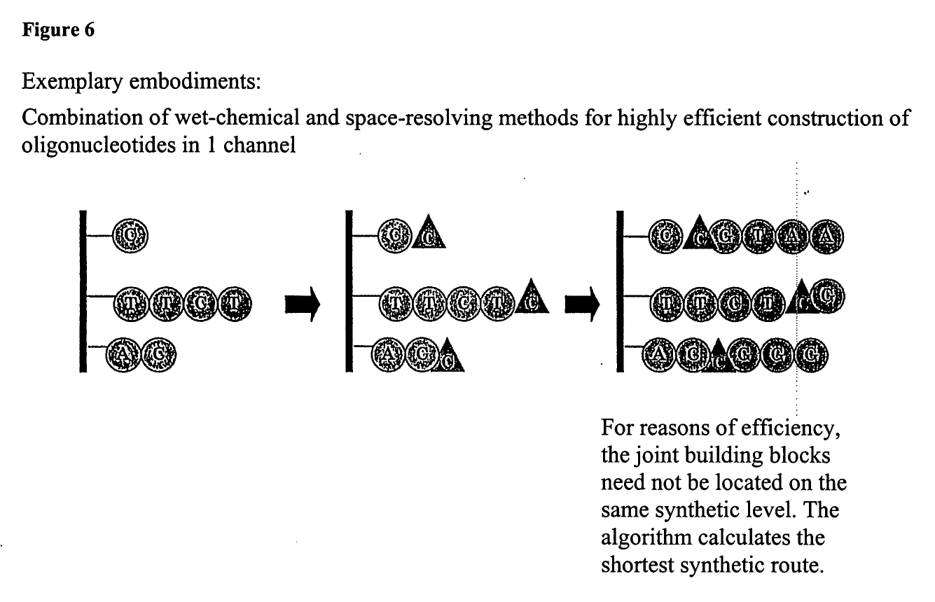

FIG. 6 shows another embodiment of the inventive combination of wet-chemical and photochemical methods; the jointly fused wet-chemical building blocks are not located on one synthetic level

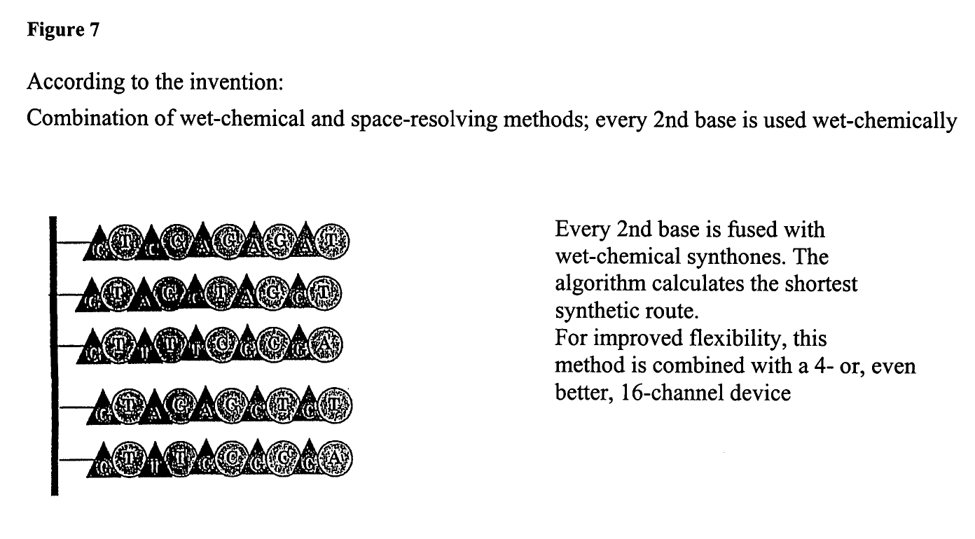

FIG. 7 shows another embodiment of the inventive combination of wet-chemical and photochemical methods; every 2nd base is fused wet-chemically

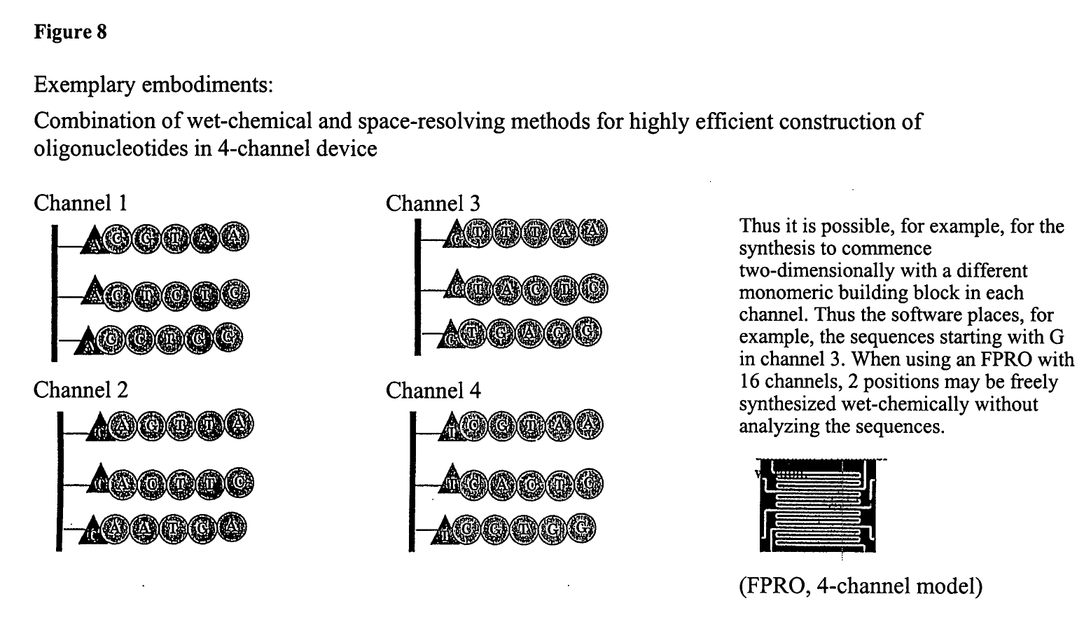

FIG. 8 shows another embodiment of the inventive combination of wet-chemical and photochemical methods in combination with a microfluidic reaction carrier with 4 channels

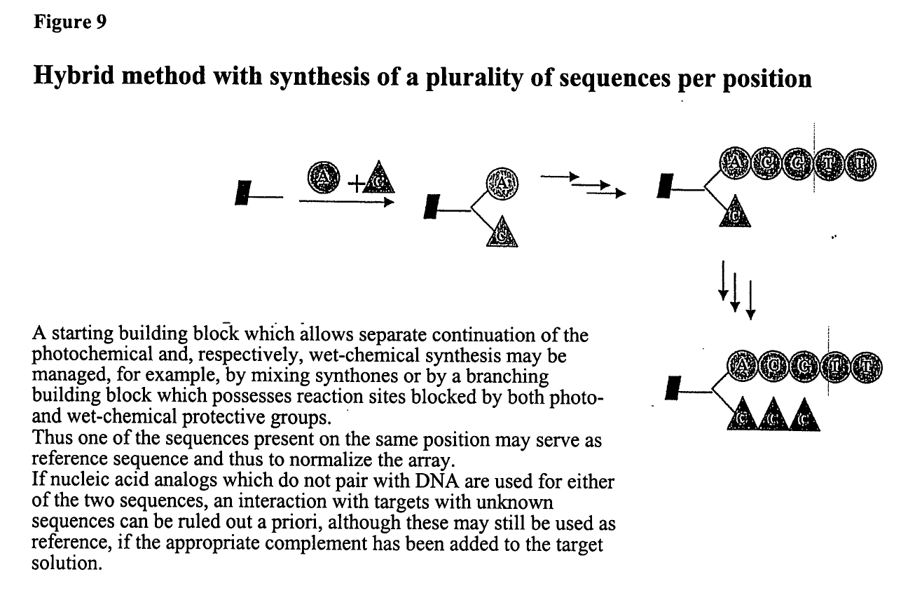

FIG. 9 shows the principle of the hybrid method with synthesis of a plurality of sequences per position with the aid of a mixture of nucleotide building blocks

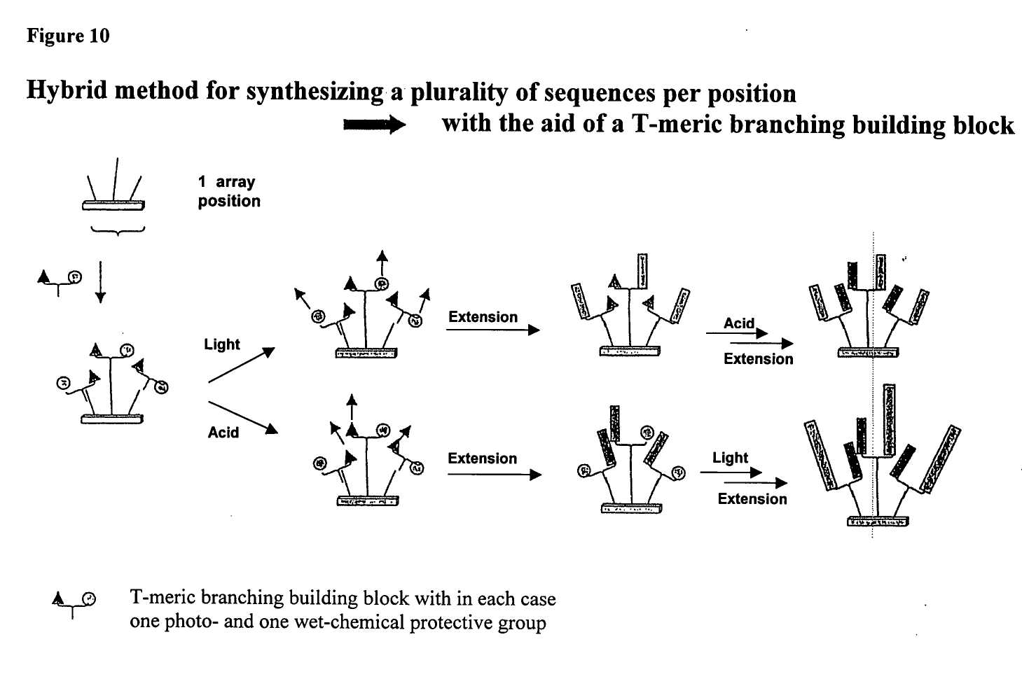

FIG. 10 shows the principle of the hybrid method with synthesis of a plurality of sequences per position with the aid of a T-meric branching building block

FIG. 11 shows the calculation of the shortest synthetic route using wet- and photochemical nucleotide building blocks

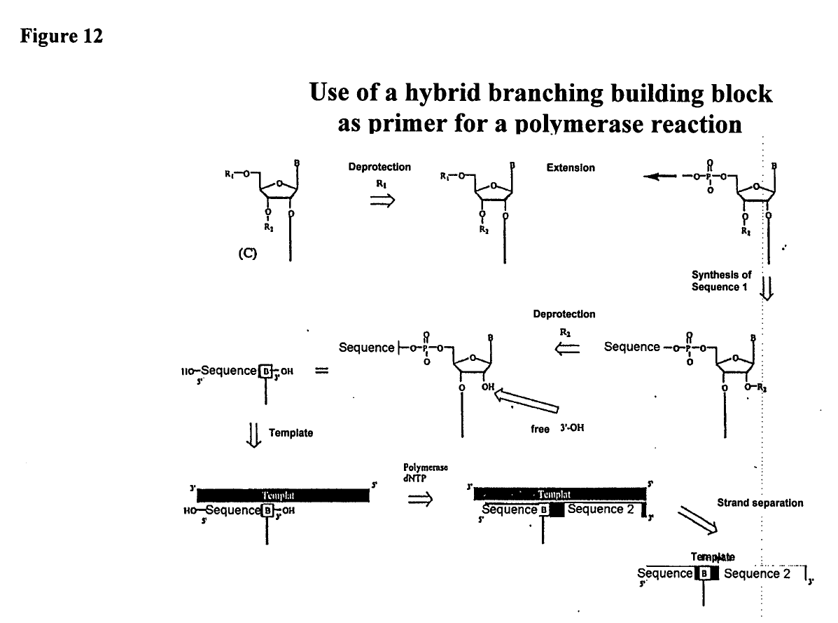

FIG. 12 shows the use of a hybrid branching building block as primer for a polymerase reaction.

1. A method for producing a carrier for the determination of analytes, comprising the steps:
(a) providing a carrier,
(b) conducting a liquid containing building blocks for the synthesis of polymeric receptors over the carrier,
(c) immobilizing in a location- or/and time-specific manner the receptor building blocks in in each case predetermined regions on said carrier and
(d) repeating steps (b) and (c), until the desired receptors have been synthesized in the in each case predetermined regions,
characterized in that
synthesis of said receptors comprises a combination of wet-chemical and photochemical synthesis steps.

2. The method as claimed in claim 1,
characterized in that
a microfluidic carrier with channels, preferably with closed channels, in which predetermined regions with immobilized receptors are generated, is used.

3. The method as claimed in claim 1 or 2,
characterized in that
the receptors are selected from biopolymers such as, for example, nucleic acids, nucleic acid analogs, proteins, peptides and carbohydrates.

4. The method as claimed in any of claims 1 to 3,
characterized in that
the receptors are selected from nucleic acids and nucleic acid analogs.

5. The method as claimed in claim 4,
characterized in that
synthesis of the receptors comprises using a combination of wet-chemical phosphoramidite building blocks and of photochemical phosphoramidite building blocks.

6. The method as claimed in any of claims 1 to 5,
characterized in that
a carrier with a plurality of, preferably with at least 50, and particularly preferably with at least 100, different receptor regions is produced.

7. The method as claimed in any of claims 1 to 6,
characterized in that
the receptors contain in one region of the carrier a single sequence of building blocks.

8. The method as claimed in any of claims 1 to 6,
characterized in that
the receptors in one region of the carrier contain a plurality of different sequences of building blocks.

9. The method as claimed in claim 8,
characterized in that
the receptors in one region of the carrier comprise reference sequences and analyte determination sequences.

10. The method as claimed in any of claims 1 to 9,
characterized in that
synthetic building blocks are used which carry a wet-chemical and a photochemical protective group.

11. The method as claimed in any of claims 1 to 10,
characterized in that
the carrier is used in situ for an analyte determination process.
